(19) United States
(12) Plant Patent Application Publication
(10) Pub. No.: US 2010/0107287 P1
(43) Pub. Date: Apr. 29, 2010

(54) Almond tree named 'Matan'

(50) ;

(75) Inventors: Doron Holland, Hasolelim (IL); Irit Bar-Ya'akov, Qiryat Tivon (IL); Kamel Hatib, Arrabe (IL); Reuven Birger, Kfar Kish (IL)

Correspondence Address: FOLEY AND LARDNER LLP;SUITE 500, 3000 K STREET NW, WASHINGTON, DC, 20007, US

(73) Assignee: STATE OF ISRAEL, MINISTRY OF AGRICULTURE & RURAL DEVELOPMENT, AGRICULTURAL RESEARCH ORGANIZATION

(21) Appl. No.: 12/589,687

(22) Filed: Oct. 27, 2009

(30) Foreign Application Priority Data
Oct. 29, 2008 (IL) 4169/08

(51) Int. Cl. A01H 5/00

(52) U.S. Cl. PLT/155

(57) ABSTRACT

A new and distinct almond variety of Prunus dulcis named ‘MATAN’, particularly characterized by self-fertile capability and production of high quality almond fruit with a large kernel.

### PRIORITY CLAIM

This application claims priority under 35 U.S.C. § 119(f) of the Israeli Plant Breeders' Rights Application No. 4169/08 filed Oct. 29, 2008.

### LATIN NAME OF THE GENUS AND SPECIES OF THE PLANT CLAIMED

Prunus dulcis

### VARIETY DENOMINATION

‘MATAN’

### BACKGROUND OF THE INVENTION

The present invention relates to a new and distinct variety of almond tree, botanically known as Prunus dulcis, synonymous with Prunus amygdalus Batsch., Amygdalus communis L., and Amygdalus dulcis Mill., belongs to the Rosaceae family, and is hereinafter referred to by the variety denomination ‘MATAN’.

The new Prunus dulcis ‘MATAN’ is a product of a controlled breeding program conducted by the inventors, Doron Holland, Irit Bar-Ya'akov, Kamel Hatib and Reuven Birger in Yizre'el Valley, Israel. The objective of the breeding program was to develop a new Prunus dulcis variety which is self fertile and produces almond fruit, of high quality and with a large kernel.

The new Prunus dulcis ‘MATAN’ originated from a cross made by the inventors in 2001 in Yizre'el Valley, Israel. The female or seed parent is the French, self-fertile Prunus dulcis variety designated Lauranne, (unpatented), and the male or pollen parent is the local Prunus dulcis variety designated ‘Um El Fahem’ (unpatented). The new Prunus dulcis ‘MATAN’ was selected by the inventors from the progeny of the stated cross in a controlled environment in 2005 in Yizre'el Valley, Israel.

Asexual propagation of the new Prunus dulcis ‘MATAN’ by grafting onto the unpatented (Prunus persica (L.) batsch×Prunus amygdalus batsch rootstock designated GF.677 was first performed in March of 2005 (two trees) and then in February of 2006 (six trees) in the orchard located in Yizre'el Valley, Israel. Asexual propagation of the new Prunus dulcis ‘MATAN’ has demonstrated that the combination of characteristics as herein disclosed for the new variety are firmly fixed and retained through successive generations of asexual propagation. The new variety reproduces true to type.

### BRIEF DESCRIPTION OF THE INVENTION

The following traits have been repeatedly observed and are determined to be unique characteristics of ‘MATAN’ which in combination distinguish this almond tree as a new and distinct variety:
1. self-fertile capability;
2. production of high quality almond fruit; and
3. large kernel of almond fruit.

In comparison to the parental varieties, ‘Lauranne’ (unpatented) and ‘Um El Fahem’ (unpatented), ‘MATAN’ differs primarily in the traits listed in Table 1.

TABLE 1

| | | Female Parent | Male Parent |
|---|---|---|---|
| | New Variety | ‘Lauranne’ | ‘Um El Fahem’ |
| Trait | ‘MATAN’ | (unpatented) | (unpatented) |
| Self-fertilization | Able | Able | Unable |
| Beginning of | February | March | February |
| Flowering | | | |
| Time of | July | August | July |
| Ripening | | | |
| Dry Fruit shell | Semi-Hard, | Hard, Complete | Soft |
| | Breakable | | |
| Taste | Sweet | Slightly Bitter | Sweet |

Among the many commercial varieties known to the present inventors, the variety most similar to ‘MATAN’ is the female parent, ‘Laurane’, as described in the foregoing Table 1.

### BRIEF DESCRIPTION OF THE PHOTOGRAPHS

The accompanying photographs illustrate the overall appearance of the new Prunus dulcis ‘MATAN’ showing the colors as true as is reasonably possible with colored reproductions of this type. Colors in the photographs may differ slightly from the color values cited in the detailed botanical description, which accurately describe the color of ‘MATAN’.

FIG. 1, taken on May, 13, 2009, shows a side-view perspective of a fruit-bearing tree of ‘MATAN’, at four year old age.

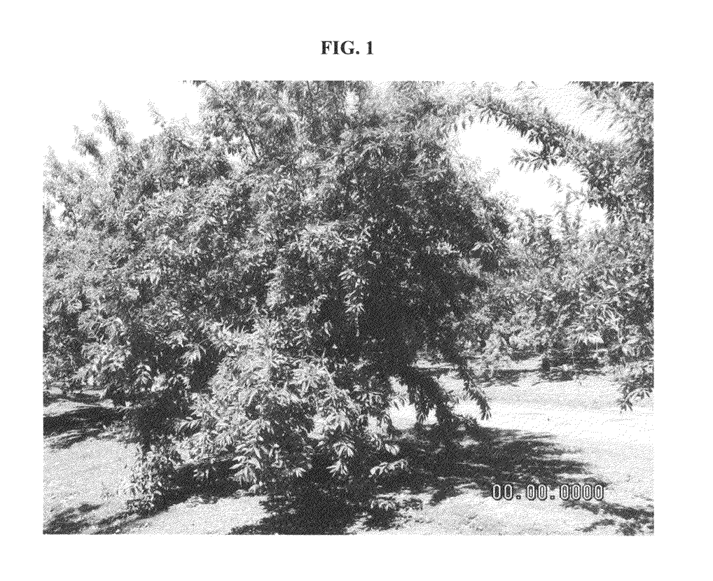

FIG. 2, taken on April, 20, 2006, shows a close-up view of a fruit-bearing tree of ‘MATAN’, at four year old age.

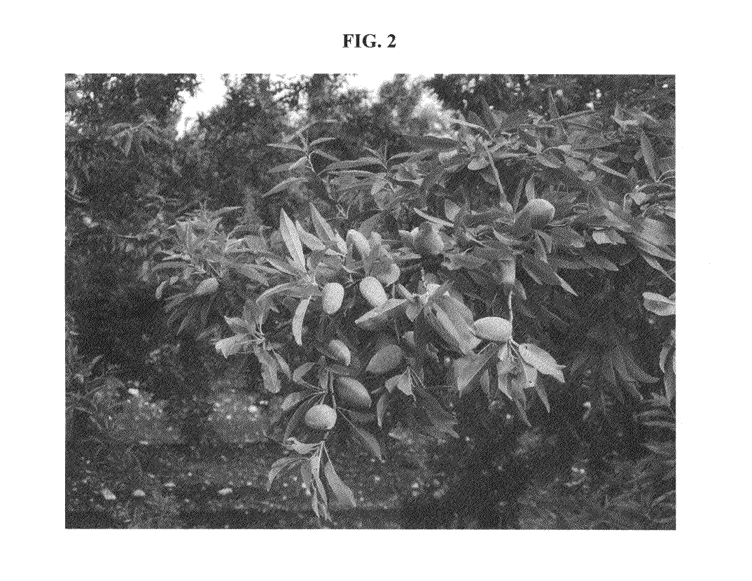

FIG. 3 shows different close-up images of typical dry fruit of ‘MATAN’.

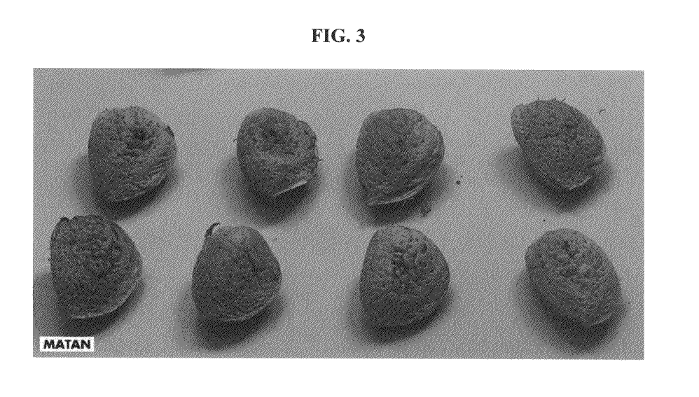

FIG. 4 shows different close-up images of typical kernels of mature fruit of ‘MATAN’

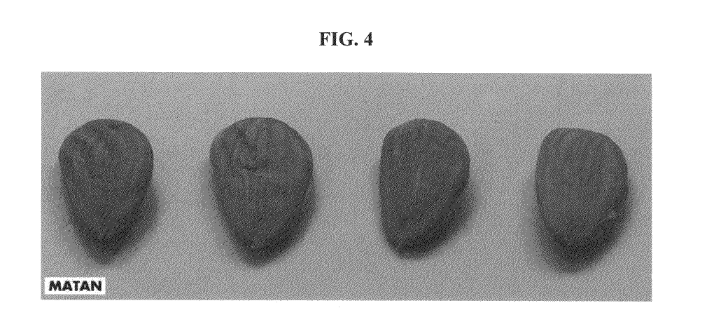

### DETAILED BOTANICAL DESCRIPTION

The new Prunus dulcis ‘MATAN’ has not been observed under all possible environmental conditions. The phenotype of the new variety may vary with variations in environment such as temperature, light intensity, and day length without any change in the genotype of the almond tree.

The aforementioned photographs, together with the following observations, measurements and values describe trees of ‘MATAN’ as grown in Yizre'el Valley, Israel, in an open orchard under conditions which closely approximate those generally used in commercial practice. The trees of ‘MATAN’ were planted mainly in clay grumusol (vertisol) soil at an elevation of about 100 meters above sea level, at latitude 32°42′N, longitude 35°11′E. Yizre'el Valley is characterized by a Mediterranean subtropical climate, with an average annual rainfall of about 580 mm concentrated from November through March. Mean diurnal minimum temperature in January is 6° C. (43° F.), and mean diurnal maximum temperature in July is 33° C. (91° F.). Fertilization of about 250 kg/ha N and 300 kg/ha K is administered per year.

Unless otherwise stated, the detailed botanical description includes observations, measurements and values based on four year old ‘MATAN’ trees grown in Yizre'el Valley, Israel from 2006 to 2009. Quantified measurements are expressed as an average of measurements taken from a number of trees of ‘MATAN’. The measurements of any individual tree, or any group of trees, of the new variety may vary from the stated average.

Color references are made to The Royal Horticultural Society Colour Chart (R.H.S.), 1986 edition, except where general colors of ordinary significance are used. Color values were taken under conditions of full sunlight.

All of the trees of ‘MATAN’, insofar as they have been observed, have been identical in all the characteristics described below.
Classification:
   Botanical.—Prunus dulcis.
Parentage:
Female or seed parent: Prunus dulcis ‘Lauranne’ (unpatented)
Male or pollen parent: Prunus dulcis ‘Um El Fahem’ (unpatented)
Propagation: Grafting onto unpatented (Prunus persica×Prunus amygdalus) rootstock designated GF.677.
Growing conditions:
Light intensities: Full sunlight.
Temperature: Mean diurnal minimum temperature in January is 6° C. (43° F.), and mean
Fertilization: About 250 kg/ha N and 300 kg/ha K is administered per year.
Growth regulators: No growth regulators are used.
Pruning or trimming requirements: No pruning or trimming applied.
Tree:
Age: Observed trees were 4 years old.
Vigor: Strong
Form: Upright, moderately dense, spreading canopy with narrow branch angles.
Habit: A medium-sized tree with one trunk and 4-5 main branches; main branches spreading; crown symmetrical and round in shape.
Branching habit: Main branches angle is 30° to 60° with respect to trunk if allowed to grow naturally.
Cropping behavior: Production of dry fruit starts and ends during first 2 weeks of August in Yizre'el Valley, Israel.
Type of bearing: Fruit set on branches mainly on distal part of branch.
Production per year: Fruit production depends on fruit set which depends on prevailing weather conditions and subsequent development of fruit.
Size of tree:
   Height.—About 3 m.
   Spread.—About 3 m.
Trunk:
   Height.—About 80 cm.
   Diameter.—(at 70 cm above ground) About 15 cm.
Main branches:
   Number per tree.—About 4-5.
   Length.—Varies due to round shape of tree; maximum of about 2.5 m; minimum of about 2 m. On average, branches grow about 50 cm to 60 cm per growing season, and then new branchlets form, radiating from the older, primary branch.
   Diameter (at 4 years).—About 5 mm to 10 mm.
   Pubescence.—None.
Spur: Present.
Foliage:
Arrangement: Alternate.
Lamina:
   Size.—Length: About 90 mm to 100 mm Width: About 20 mm to 30 mm Length/Width ration: 0.2 to 0.33.
   Overall shape.—Elliptic.
   Base shape.—Acute.
   Apex shape.—Acute.
   Margin.—Crenate.
   Margin undulation.—None or slight.
   Texture.—Upper surface: Glabrous.
   Under surface.—Glabrous.
Venation:
   Type.—Pinnate venation from central vein to the leaf edge.
Petiole:
   Length.—About 20 mm.
   Diameter.—About 1 mm.
   Texture.—Smooth.
Stipule: None
Glands: None
Flowers:
Blooming period: Up to 10 days depending on weather conditions.
Fragrance: None or vary slight
Type: Flowers are born single or clustered with 2-3 per cluster on branches and on spurs.
Petals:
   Number per flower.—Five.
Sepals:
   Number per flower.—Five.
Fruit:
Keeping quality: Dried fruit and nuts can be stored for over 1 year.
Maturity when described: Ripe for eating.
Maturity period after full bloom: Beginning of August in Yizre'el Valley.
Date of first and last picking (harvest): About August 1^{st} and August 15^{th} in Newe Yaar, Israel.
Distribution on tree: Well distributed throughout.
Type: Drupe.
Length (along suture): About 45 mm to 50 mm
Width: About 30 mm to 35 mm
Immature hull:
   Pubescence.—Abundant (strong).
Mature in-shell nut:
   Overall shape.—Elongated.
   Shape at apex.—Right angle to mucron.
   Length.—About 37 mm to 45 mm.
   Width.—About 24 mm to 28 mm.
   Thickness.—About 15 mm to 20 mm.
   Weight.—About 6.5 g to 7.5 g.
   Thickness of endocarp.—Thin.
   Resistance to cracking.—Weak.
   Left over hull.—Present.
   Keel development.—Strong.
   Color.—Brown, closest to greyed-orange group, RHS 164C.
Kernel:
   Overall shape.—Ovate.
   Apex shape.—Obtuse.
   Base shape.—Truncate to rounded.
   Size.—Large.
   Length.—About 25 mm to 30 mm.
   Width.—About 14 mm to 17 mm.
   Thickness.—About 6 mm to 7 mm.
   Weight.—About 1.6 g.
   Rugosity.—Medium roughness.
   Pubescence.—None.
   Color.—Brownish-red, closest to greyed-orange group, RHS 164A.
   Number of doubles.—None.
Flavor: Sweet
Eating quality: Excellent
Market: Fresh market.
Disease/pest resistance: No atypical resistance has been noted.
Disease/pest susceptibility: No atypical resistance has been noted.
Winter hardiness: Tolerant to temperatures down to −9° C. without observed damage to wood and buds of dormant almond trees.
Drought/heat tolerance.Tolerant to temperatures up to 33° C., growth is limited by drought periods without irrigation.

What is claimed is:

1. A new and distinct almond variety of Prunus dulcis named ‘MATAN’, as illustrated and described herein.
